(19) United States
(12) Patent Application Publication
(10) Pub. No.: US 2018/0358113 A1
(43) Pub. Date: Dec. 13, 2018

(54) TWO-FACTOR AUTHENTICATION IN A PULSE OXIMETRY SYSTEM

(71) Applicant: KONINKLIJKE PHILIPS N.V., EINDHOVEN (NL)

(72) Inventors: John CRONIN, BONITA SPRINGS, FL (US); Michael D'ANDREA, BONITA SPRINGS, FL (US)

(21) Appl. No.: 15/777,270

(22) PCT Filed: Nov. 16, 2016
(86) PCT No.: PCT/EP2016/077787

§ 371 Date: May 18, 2018

Related U.S. Application Data
(60) Provisional application No. 62/259,057, filed on Nov. 24, 2015.

(30) Foreign Application Priority Data
Mar. 4, 2016 (EP) 16158763.9

(51) Int. Cl. G16H 10/60; G06F 21/32; G16H 50/30

(52) U.S. Cl. CPC G16H 10/60; G16H 50/30; G06F 21/32

(57) ABSTRACT

Systems and methods for patient identification and health monitoring. The system of the present invention comprises a pulse oximeter, a second physiological sensor, a remote server, and a virtual private network. The pulse oximeter comprises a pulse oximeter's biometric reader, which acquires a biometric data and determines if the acquired biometric data matches a corresponding previously stored biometric template. If a match is determined, the pulse oximeter executes a second authentication scheme to acquire a second authentication data, which is transmitted via a virtual private network to a remote server. The remote server then determines if the transmitted second authentication data matches a registered authentication data. If a match is determined, a patient is identified.

### BACKGROUND OF THE INVENTION

Many computer-based diagnostic systems allow sharing of various patient-related information such as family medical history, allergies, pre-existing medical conditions, including treatment protocols that a patient is undergoing. Thus, it is important to implement data integrity and security systems in hospital computer-based diagnostic systems to not only prevent data theft or security breach, but to also ensure data accuracy and consistency when transmitting data from one point to another. With those systems in place, the medical personnel can rely on the accuracy of recorded physiological data that the medical personnel will use to arrive at a correct patient diagnosis.

Security systems typically implement a variety of authentication schemes, which can either be one, two, or multi-factor authentication, to improve data privacy such that access to patient's medical information is limited to a list of authorized individuals. In addition to requiring authentication procedures, various data encryption algorithms can also be used so that only computer-based diagnostic systems with the correct cryptographic key can decrypt a patient's information.

U.S. patent application number 2009/0043180 discloses a pulse oximeter integrated with a fingerprint sensor connected to one or more physiological sensors, such as a heart rate sensor and temperature sensor, for acquiring patient health data. When a patient's fingerprint is identified, the acquired patient health data is then associated with the patient's medical records. U.S. patent application number 2006/0074280 discloses a patient identification device that comprises a patient identifier and a pulse oximeter. Patient identification can be performed through biometrics authentication such as fingerprint identification, retinal identification, and pulse oximetry data comparison.

### SUMMARY OF THE INVENTION

The present invention relates to systems and methods for patient identification and health monitoring. The system of the present invention comprises a pulse oximeter, a second physiological sensor, a patients database, a remote server, and a virtual private network. The pulse oximeter comprises a pulse oximeter's biometric reader, a pulse oximeter processor, a pulse oximeter memory, and a communication module. The method of the present invention comprises acquiring via a pulse oximeter's biometric reader a biometric data and identifying a patient by matching the acquired biometric data with a corresponding previously stored biometric template. If there is a match, the pulse oximeter executes a second authentication scheme to acquire a second authentication data, which is transmitted via a virtual private network to a remote server. The remote server then determines if the transmitted second authentication data matches a registered authentication data. If a match is determined, remote server triggers the pulse oximeter and second physiological sensor to acquire physiological data.

The present invention relates to a method for patient identification and health monitoring comprising: acquiring via a pulse oximeter's biometric reader a biometric data; identifying a patient by matching the acquired biometric data with a corresponding previously stored biometric template in the pulse oximeter memory, wherein the corresponding previously stored biometric template is generated by encrypting a previously acquired biometric data; acquiring a second authentication data using a second authentication scheme when the acquired biometric data matches with the corresponding previously stored biometric template; transmitting via a virtual private network the acquired second authentication data to a remote server; determining by the remote server if the transmitted second authentication data matches a registered authentication data; triggering by the remote server the pulse oximeter and a second physiological sensor to acquire physiological data when the transmitted second authentication data matches the registered authentication data, wherein the acquired physiological data comprise pulse oximetry data and second physiological sensor data; acquiring physiological data using the pulse oximeter and the second physiological sensor; and storing the acquired physiological data in a database.

The present invention also relates to a system for patient identification and health monitoring comprising: a pulse oximeter for acquiring pulse oximetry data. The pulse oximeter comprises: a pulse oximeter's biometric reader for acquiring a biometric data and a pulse oximeter processor for matching the acquired biometric data to a corresponding previously stored biometric template, wherein the corresponding previously stored biometric template is generated by encrypting a previously acquired biometric data. The pulse oximeter processor also executes a second authentication scheme to acquire a second authentication data when the acquired biometric data matches with the corresponding previously stored biometric template. The pulse oximeter further comprises a pulse oximeter memory for storing the acquired biometric data, the corresponding previously stored biometric template, the acquired second authentication data, and the acquired pulse oximetry data. The pulse oximeter also further comprises a communication module for transmitting to a remote server the acquired second authentication data and to a patients database the acquired pulse oximetry data. The system further comprises a second physiological sensor for acquiring a second physiological sensor data; a patients database for storing the transmitted pulse oximetry data and the acquired second physiological sensor data; and a remote server for determining if the transmitted second authentication data matches a registered authentication data. The remote server also triggers the pulse oximeter and a second physiological sensor to acquire physiological data when the transmitted second authentication data matches the registered authentication data, wherein the acquired physiological data comprise the pulse oximetry data and the second physiological sensor data. The system also further comprises a virtual private network for allowing secure data communication among the pulse oximeter, the cloud network, and the remote server.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention.

In the drawings:

FIG. 1 illustrates a system for patient identification and health monitoring according to a preferred embodiment of the present invention.

FIG. 2 illustrates a method for patient identification and health monitoring according to a preferred embodiment of the present invention.

FIG. 3 illustrates a graphical user interface for authenticating a patient's fingerprint data according to a preferred embodiment of the present invention.

FIG. 4A and FIG. 4B illustrate a method for patient authentication according to another preferred embodiment of the present invention.

### DETAILED DESCRIPTION OF THE EMBODIMENTS

The following are definitions of terms as used in the various embodiments of the present invention.

The term “second physiological sensor” as used herein refers to any device, instrument, equipment, or apparatus capable of measuring a physiological parameter or assisting in the diagnosis of a physiological condition or disease. Examples of second physiological sensors are body temperature sensors, galvanic skin response sensors, and other sensors capable of detecting electrocardiograph patterns, heart rate, blood alcohol content, respiratory rate, and glucose level.

The term “biometric template” as used herein refers to a digital template based on distinct characteristics or feature extracted from a biometric data such as fingerprint image data, voice data, face image data, iris-scanned image data, retina-scanned image data, vein pattern data, and hand geometry data that includes a 3D image of top and sides of hand and fingers. The biometric template is generated by encrypting a biometric data acquired from a patient. The biometric template is used during biometric authentication process by comparing a verification template with a corresponding previously stored biometric template.

The term “verification template” as used herein refers to a biometric template for authenticating a person's identity by comparing the verification template with a corresponding previously stored biometric template.

The term “database” as used herein refers to a collection of data and information organized in such a way as to allow the data and information to be stored, retrieved, updated, and manipulated and to allow them to be presented into one or more formats such as in table form or to be grouped into text, numbers, images, and audio data. The term “database” as used herein may also refer to a portion of a larger database, which in this case forms a type of database within a database. “Database” as used herein also refers to conventional databases that may reside locally or that may be accessed from a remote location, e.g., remote network servers. The database typically resides in computer memory that includes various types of volatile and non-volatile computer memory. Memory wherein the database resides may include high-speed random access memory or non-volatile memory such as magnetic disk storage devices, optical storage devices, and flash memory. Memory where the database resides may also comprise one or more software for processing and organizing data received by and stored into the database.

The term “patients database” as used herein refers to a database comprising patient data corresponding to patient's personal information, physiological data, medical diagnoses, and medicines and treatments the patient is being presently administered to.

The term “authentication database” as used herein refers to a database comprising authentication information used as reference data such as biometric template, signature-scanned image data, keystroke data, and password.

The term “virtual private network” as used herein refers to a private network that securely connects remote computers or computer networks at different locations via the Internet. The virtual private network uses various data encryption and other security protocols to restrict data access within a network to authorized computers. This ensures, for example, that the patient's medical data and authentication data cannot be intercepted and decrypted during data communication between a user computer and a remote server.

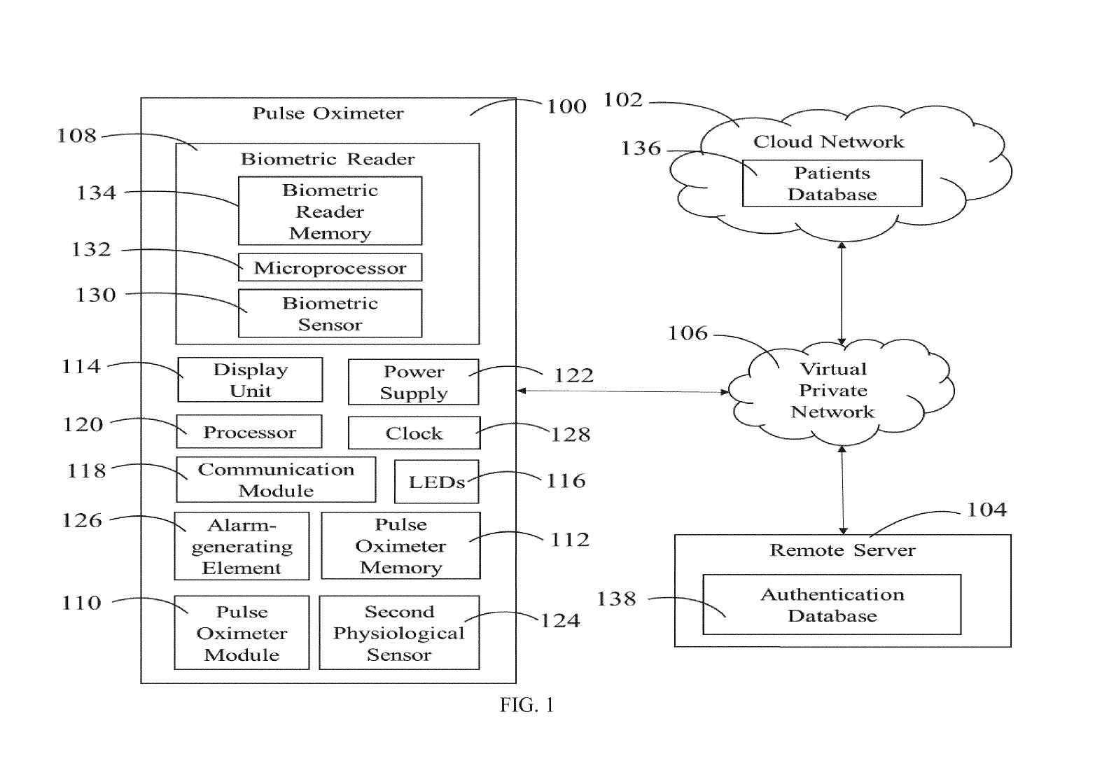

In a preferred embodiment of the present invention illustrated in FIG. 1, a system for patient identification and health monitoring comprises a pulse oximeter 100, a cloud network 102, a remote server 104, and a virtual private network 106. The pulse oximeter 100 comprises a pulse oximeter biometric reader 108, a pulse oximeter module 110, a pulse oximeter memory 112, a display unit 114, one or more LEDs 116, a communication module 118, a processor 120, a power supply 122, a second physiological sensor 124, an alarm-generating element 126, and a clock 128. The pulse oximeter biometric reader 108 comprises a biometric sensor 130, a microprocessor 132, and a biometric reader memory 134. The cloud network 102 comprises a patients database 136. The remote server 104 comprises an authentication database 138.

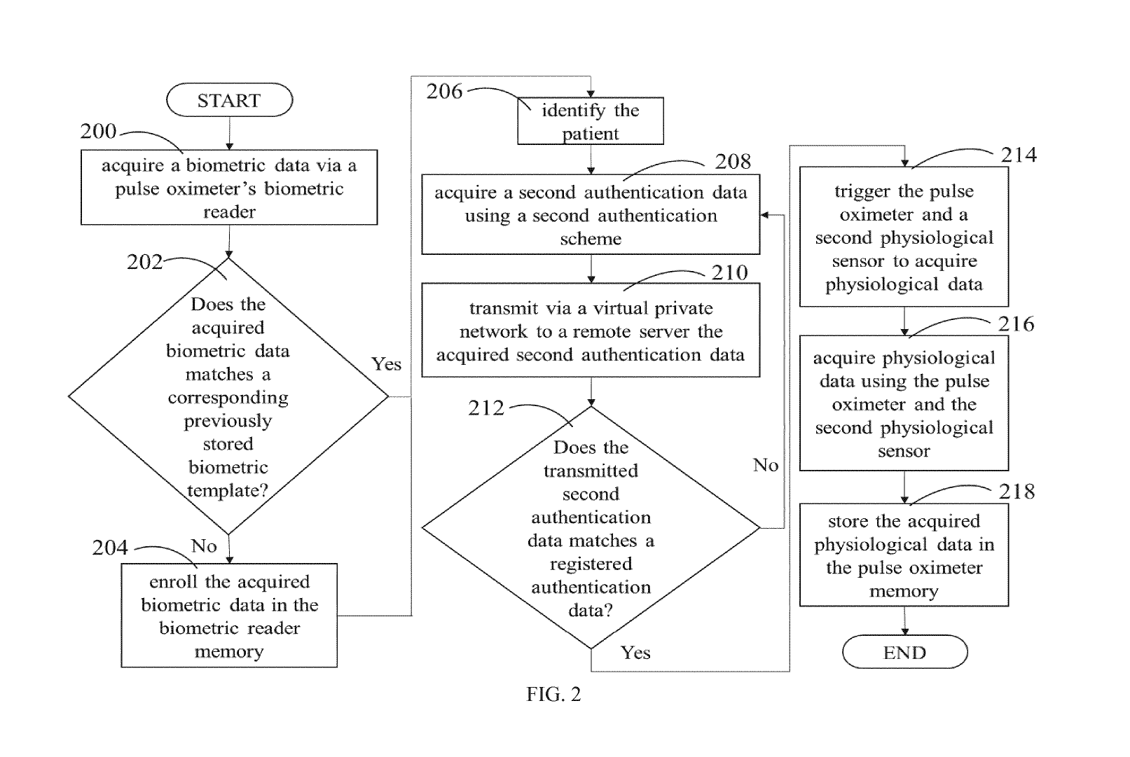

FIG. 2 illustrates a preferred method of the present invention. A patient complaining of itching, body and face swelling, and breathing difficulty is brought to a hospital. A nurse assists the patient in authenticating the patient's identity before acquiring physiological data such as blood oxygen saturation level, body temperature, and pulse rate. The nurse uses the pulse oximeter biometric reader to acquire the patient's biometric data—for example, fingerprint image data, voice data, face image data, iris-scanned image data, retina-scanned image data, signature-scanned image data, keystroke data, hand geometry data that includes a 3D image of top and sides of hand and fingers—from the patient (step 200). Preferably, the acquired biometric data is sampled to extract a subset of biometric data points representative of the patient's biometric features (e.g., location of fingerprint's ridge endings and bifurcations) so that the acquired biometric data block size is decreased. After sampling the acquired biometric data, the extracted subset of biometric data points are encrypted using one or more encryption techniques, such as Advanced Encryption Standard (AES), which may use different cryptographic key length (e.g., 128 bits, 192 bits, and 256 bits) to generate a verification template. The biometric reader's microprocessor then verifies if the generated verification template matches a corresponding previously stored biometric template in the biometric reader memory by calculating a score for determining a degree of similarity between the two templates (step 202). If the calculated score exceeds a predefined threshold, a match is verified.

If no match is verified, the acquired biometric data is encrypted to generate a biometric template, which is then enrolled and stored in the biometric reader memory for future biometric template comparisons (step 204). If a match is verified, the patient's identity is determined (step 206). The pulse oximeter processor then performs a second authentication scheme to acquire a second authentication data, for example, an input password, in-air signature, or a different type of biometric data from the first acquired biometric data, (step 208). Thereafter, the second authentication data is transmitted via a virtual private network to a remote server (step 210). Preferably, the second authentication data is encrypted first before being transmitted to the remote server. Upon receiving the encrypted second authentication data, the remote server decrypts the second authentication data from the encrypted second authentication data. In this preferred embodiment, a private key is shared in advance prior to communication between the pulse oximeter and the remote server to allow correct encryption and decryption of data being communicated.

After decryption, the remote server checks if the second authentication data matches a registered authentication data in the remote server's authentication database (step 212). If there is a match, the remote server sends a signal for triggering the pulse oximeter and second physiological sensor to acquire physiological data, wherein the physiological data comprise the pulse oximetry data and second physiological sensor data (step 214). The pulse oximeter and second physiological sensor then acquires physiological data (step 216) to be stored in the pulse oximeter memory (step 218). Preferably, the acquired physiological data is transmitted via the virtual private network and stored in the cloud network's patients database. If the second authentication data does not match the registered authentication data or if an error (e.g., computer or human error) occurred during data acquisition, the patient is prompted to acquire an additional second authentication data.

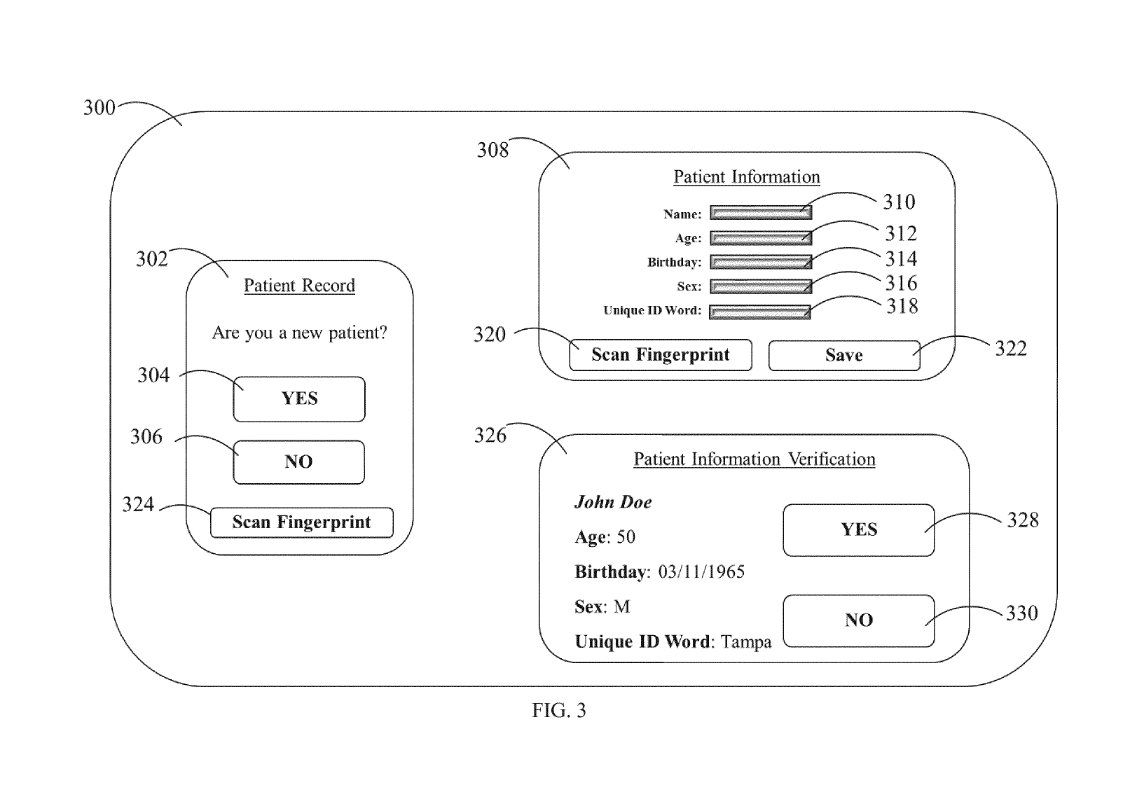

In a preferred embodiment of the present invention illustrated in FIG. 3, a pulse oximeter's graphical user interface 300 allows a patient to authenticate his fingerprint image data. The question “Are you a new patient?” is displayed on the graphical user interface's “Patient Record” window 302 to the patient who answers the question by selecting either the “YES” 304 or “NO” button 306. If the patient selects the “YES” button 304, the patient is given the option to access the “Patient Information” window 308, which requires the patient to input the corresponding information on the following text entry boxes: “Name” text entry box 310, “Age” text entry box 312, “Birthday” text entry box 314, “Sex” text entry box 316, and “Unique ID Word” text entry box 318. The “Patient Information” window 308 also displays to the patient a “Scan Fingerprint” button 320, which gives the patient an access to scanning his fingerprint using the pulse oximeter's fingerprint scanner. After the patient scans his fingerprint and verifies that all the inputted information on the required text entry boxes are correct, the patient is provided with a “save” button 322 to create a new patient medical record in the hospital's main computer system. In this embodiment, the patient preferably scans and saves at least two fingerprint image data so that when one of the patient's fingerprints (e.g., right hand's index fingerprint) cannot be recognized by the pulse oximeter's fingerprint scanner (e.g., due to skin peeling and cracks on the finger's surface), the other fingerprint image data can be used for authentication. Here, the patient's fingerprint image data serves as the first authentication reference data while the unique ID word entered by the patient serves as the second authentication reference data.

On the same graphical user interface 300 shown in FIG. 3, if the patient answers the question “Are you a new patient?” by choosing the “NO” button 306, the patient may select the “Scan Fingerprint” button 324 to initiate the pulse oximeter's fingerprint scanner. The patient scans his fingerprint and, when the patient's fingerprint is recognized, the graphical user interface opens the “Patient Information Verification” window 326 that allows the patient to verify (i.e. by pressing either the “Yes” 328 or “No” button 330) if the displayed information are correct.

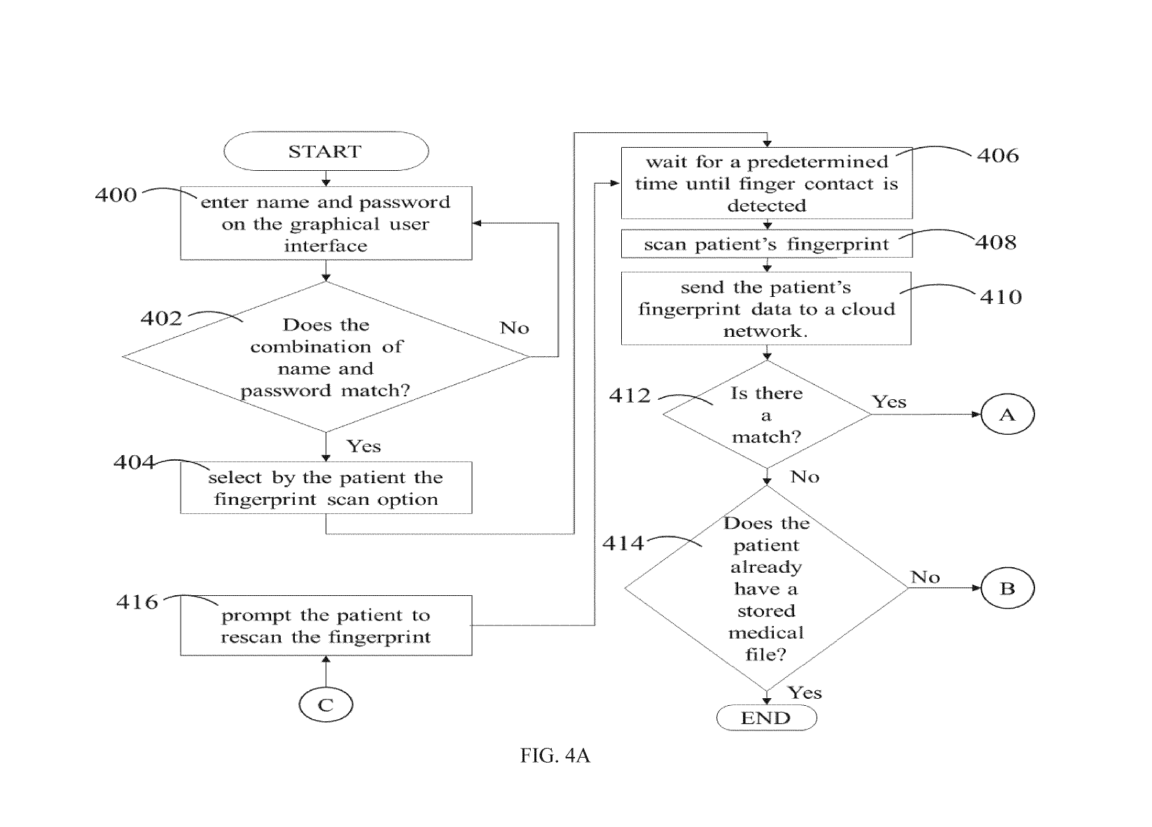

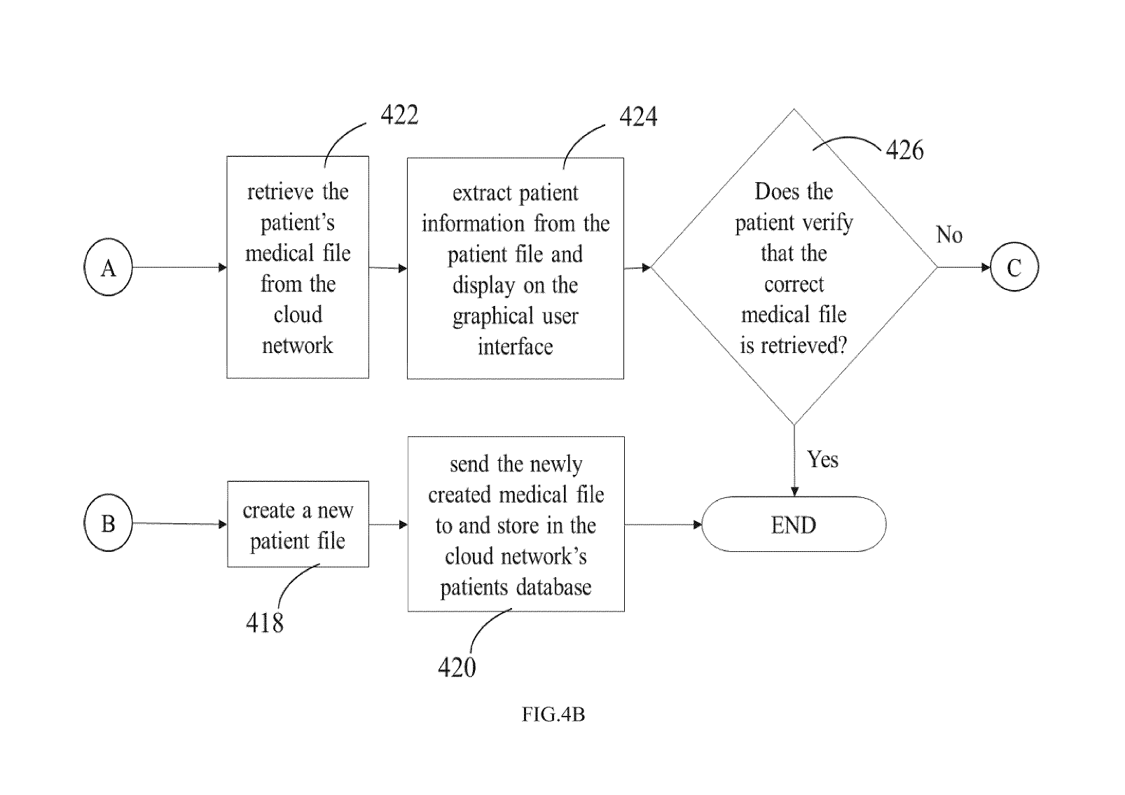

FIG. 4A and FIG. 4B illustrate a preferred embodiment of the present invention relating to a method for patient authentication. A patient enters a health clinic to attend a scheduled medical consultation. Before the patient can consult the attending physician, the patient is requested by a nurse to retrieve the patient's medical file to be presented to the doctor. To successfully retrieve the patient's medical file, the patient must be authenticated first using a two-factor authentication scheme. The patient enters his name and password via a pulse oximeter graphical user interface (step 400). The pulse oximeter then determines if the combination of the name and password entered by the patient matches a corresponding reference data stored in the cloud network's patients database (step 402). If the combination does not match, the patient is requested to re-enter his name and password. If a match is found, the patient accesses the pulse oximeter's graphical user interface to select the fingerprint scan option (step 404). The pulse oximeter's fingerprint scanner is then instructed to wait for a predetermined time until finger contact is detected (step 406). When finger contact is detected, the pulse oximeter's fingerprint scanner scans the patient's fingerprint (step 408). The pulse oximeter then samples the patient's fingerprint data, encrypts the sampled fingerprint data, generates a fingerprint template, and sends the fingerprint template to a cloud network (step 410). The cloud network's computing engine determines if the fingerprint template matches a corresponding previously stored fingerprint template in the cloud network's patients database (step 412).

If no match is found, a question is displayed on the graphical user interface, which queries if the patient already has a medical file stored in the hospital's main computer system (step 414). Even though the patient already has an existing medical file, the patient might have improperly scanned his fingerprint and is thus prompted to redo the fingerprint scanning (step 416). The system then loops back to wait again for a predetermined time until finger contact is detected. If the patient is yet to have a medical file, the patient is requested to input his information using the graphical user interface and to scan his fingerprint to create a new medical file (step 418). The newly created medical file is then sent to and stored in the cloud network's patients database (step 420).

If the cloud network's computing engine determines that the patient's fingerprint template matches a corresponding previously stored fingerprint template, the patient's medical file is retrieved by the pulse oximeter from the cloud network (step 422). The pulse oximeter then extracts from the patient's medical file and displays the patient information on the graphical user interface (step 424) to allow the patient to verify if the retrieved medical file is correct (step 426). If the medical file corresponds to a different patient, the patient is prompted to rescan his fingerprint (step 416). If the retrieved medical file is correct, the patient may proceed with his scheduled consultation and present his medical file to his doctor.

In another embodiment of the present invention, a patient experiencing severe respiratory distress is in an ambulance en route to the nearest hospital. A paramedic performs standard operating procedure for patients suffering from severe respiratory distress by first performing airway management to help the patient breathe using an oxygen mask connected to a ventilation equipment. The paramedic then monitors the patient's vital signs using a health monitoring system comprising a pulse oximeter and other physiological sensors. Looking at the patient's vital signs displayed on the health monitoring system, the paramedic determines that the patient's heart rate went down to a very low pulse rate of 25 bpm and decides to use a defibrillator to normalize the patient's heart rhythm. The paramedic notices that the use of defibrillator has little effect on the patient's heart rate and decides to administer atropine to the patient to help stabilize the patient's heart rate. To verify whether the patient is allergic to the drug atropine, the paramedic attempts to retrieve the patient's medical record online. The paramedic places the patient's finger on the pulse oximeter's fingerprint scanner to authenticate the patient's fingerprint data and determine if the patient has an existing medical record stored in the cloud network. Finding the patient's medical record online, the paramedic tries to access the patient's medical record by saying “access medical record” to allow the health monitoring system to verify the paramedic's identity via a voice recognition module. When the health monitoring system has authenticated the paramedic's voice, it then retrieves the patient's medical record from the cloud network and displays the patient's medical record to the paramedic. The patient's medical record shows the patient is not allergic to atropine. Thus, the paramedic administers atropine to stabilize the patient's heart rate.

A patient's physiological data security is preferably enhanced by using a patient's biometric data as a cryptographic key for encrypting the patient's physiological data. In this encryption technique, biometric feature data is extracted from the patient's biometric data, for example, an iris image data. The extracted biometric feature data is then added with a random number calculated by a cryptographic algorithm to generate the cryptographic key for encrypting the patient's physiological data. The encrypted physiological data is then sent to and stored in the cloud network's patients database. When the patient passes the two-factor authentication system, the patient retrieves using, for example, a mobile device to retrieve the encrypted physiological data from the patients database. The patient's mobile device executes the same cryptographic algorithm used in encryption to generate a decryption key, which consists of a randomly generated number and the patient's biometric feature data. The decryption key is then used to extract the patient's physiological data from the encrypted physiological data. In executing the cryptographic algorithm, the discrepancy between the random numbers calculated for the encryption and decryption keys is compensated and corrected by an error-correcting code to enable complete recovery and retrieval of the patient's physiological data.

In one aspect of the present invention, the patient establishes his identity by typing his name on the pulse oximeter's graphical user interface. The microprocessor chip embedded on the pulse oximeter's biometric reader locates and accesses the patient's biometric template previously stored in the biometric reader's memory. The patient is then prompted to acquire his biometric data that is converted to a verification template. Thereafter, the biometric reader's microprocessor chip compares the verification template with the biometric template to determine a match. Rather than comparing a single verification template against thousands or even millions of biometric templates, this authentication process is faster since a single verification template is being matched against a single biometric template.

In a further embodiment of the present invention, two sets of biometric data can be combined to generate a single biometric template. Here, the two sets of biometric data can be acquired from the same person or one biometric data set is acquired from two different persons. The two sets of biometric data can also be of different types of biometric data such that, for example, one biometric data set is a patient's fingerprint image data and the other is a medical personnel's iris image data. In this example, the two sets of biometric data are sampled and filtered to extract two sets of biometric feature data using a fingerprint feature extraction algorithm and an iris feature extraction algorithm. Here, the iris feature extraction algorithm simplifies the raw iris image data and extracts only a subset of feature data (e.g., color gradient direction) representative of the iris image patterns. The two sets of extracted biometric feature data are then combined and encrypted to generate a combined biometric template. The biometric template is preferably then transmitted via a virtual private network to a remote server that matches the biometric template to a corresponding previously stored biometric template by calculating a matching score to determine a degree of similarity. If the matching score exceeds a predefined threshold value, a match is determined and, a signal is sent to the pulse oximeter and second physiological sensor to trigger physiological data acquisition. The patient's medical data is also retrieved via the virtual private network from the patients database and displayed on the patient monitor. In this way, both the presently acquired physiological data and the previously acquired physiological data (i.e. extracted from the patient's retrieved medical file) are displayed on the patient monitor to allow a medical personnel to accurately diagnose the patient's medical condition.

In another embodiment of the present invention, a medical personnel wishes to remotely monitor a patient at home. The medical personnel enters via a user device, such as a mobile phone, the patient's name and password unique to the patient. The medical personnel's user device transmits to a remote server the data corresponding to the patient's name and password. The remote server then checks if the transmitted password matches a registered password. If a match is found, the remote server preferably triggers the pulse oximeter to initiate patient authentication by activating the fingerprint scanner integrated in the pulse oximeter. The fingerprint scanner acquires fingerprint image data from the patient, converts the fingerprint image data to a fingerprint template, and determines if the patient's fingerprint template corresponds to a fingerprint template previously stored in the pulse oximeter memory. When a match is determined, the pulse oximeter is triggered to acquire pulse oximetry data from the patient. The preceding steps ensure that the pulse oximetry data to be acquired belongs to the patient that the medical personnel wishes to monitor. The acquired pulse oximetry data are then sent via a virtual private network and stored in a patients database. The pulse oximeter also may send a text message containing a hyperlink to a medical personnel's user device. Preferably, the hyperlink is a local IP address in a private network, which gives the medical personnel's user device authorization to access and view the patient's pulse oximetry data. The patient's pulse oximetry data is retrieved and then displayed on the medical personnel's user device to allow the medical personnel to diagnose the patient from a remote location.

In a preferred embodiment of the present invention, patient identification and health monitoring system includes an alert system. The alert system may send an alert to the hospital's main computer system notifying one or more medical personnel that the person presently attempting to access the patient information does not belong to the list of individuals authorized to monitor and access the patient's medical and personal data. An alert may also be sent to a medical personnel's user device to inform that either the pulse oximetry data or the second physiological sensor data is within the corresponding alert threshold ranges.

The present invention is not intended to be restricted to the several embodiments of the invention described above. Other variations that may be envisioned by those skilled in the art are intended to fall within the disclosure.

1. A method for patient identification and health monitoring, the method comprising:
monitoring biometric data via a biometric reader of a pulse oximeter;
using a microprocessor of the pulse oximeter, identifying a patient by matching the monitored biometric data with a corresponding stored biometric template in a memory of the pulse oximeter, wherein the corresponding stored biometric template comprises encrypted biometric data previously obtained from the patient;
using a microprocessor of the pulse oximeter, receiving a second authentication data using a second authentication scheme;
transmitting via a virtual private network the received second authentication data to a remote server;
determining by the remote server if the transmitted second authentication data matches a registered authentication data;
triggering by the remote server the pulse oximeter and a second physiological sensor to acquire physiological data when the transmitted second authentication data matches the registered authentication data, wherein the acquired physiological data comprise pulse oximetry data and second physiological sensor data;
acquiring physiological data using the pulse oximeter and the second physiological sensor; and
storing the acquired physiological data in a database.

2. The method of claim 1, further comprising requiring a dual authentication scheme before allowing access to the stored physiological data.

3. The method of claim 1, wherein the second authentication data is a second biometric data of a different biometric type from the acquired biometric data.

4. The method of claim 3, wherein the second biometric data is acquired from a different person.

5. The method of claim 1, wherein the corresponding stored biometric template includes at least one of fingerprint image data, voice data, face image data, iris-scanned image data, retina-scanned image data, vein pattern data, hand geometry data, and three-dimensional image data.

6. The method of claim 1, wherein the second physiological sensor is selected from the group consisting of sensors for detecting body temperature sensors, galvanic skin response, electrocardiograph, heart rate, blood alcohol content, respiratory rate, and glucose level.

7. The method of claim 1, further comprising encrypting the received second authentication data prior to transmission to the remote server.

8. A system for patient identification and health monitoring, the system comprising:
a pulse oximeter for acquiring pulse oximetry data comprising:
a pulse oximeter biometric reader for acquiring a biometric data;
a pulse oximeter processor that executes instructions stored in memory, wherein execution of the instructions by the processor:
matches the acquired biometric data to a corresponding previously stored biometric template, wherein the corresponding previously stored biometric template is generated by encrypting a previously acquired biometric data and
executes a second authentication scheme to acquire a second authentication data when the acquired biometric data matches with the
corresponding previously stored biometric template;
a pulse oximeter memory for storing the acquired biometric data, the corresponding previously stored biometric template, the acquired second authentication data, and the acquired pulse oximetry data; and
a communication module for transmitting to a remote server the acquired second authentication data and to a patients database the acquired pulse oximetry data;
a second physiological sensor for acquiring a second physiological sensor data;
a patients database for storing the transmitted pulse oximetry data and the acquired second physiological sensor data;
wherein the remote server:
determines if the transmitted second authentication data matches a registered authentication data, and
triggers the pulse oximeter and the second physiological sensor to acquire physiological data when the transmitted second authentication data matches the registered authentication data, wherein the acquired physiological data comprise the stored pulse oximetry data and the stored second physiological sensor data; and
a virtual private network for allowing secure data communication among the pulse oximeter, the patients database, and the remote server.

9. The system of claim 8, wherein a dual authentication scheme is required before allowing access to the stored physiological data.

10. The system of claim 8, wherein the second authentication data is a second biometric data of a different biometric type from the acquired biometric data.

11. The system of claim 10, wherein the second biometric data is acquired from a different person.

12. The system of claim 8, wherein the corresponding stored biometric template includes at least one of fingerprint image data, voice data, face image data, iris-scanned image data, retina-scanned image data, vein pattern data, hand geometry data, and three-dimensional image data.

13. The system of claim 8, wherein the second physiological sensor is selected from the group consisting of sensors for detecting body temperature sensors, galvanic skin response, electrocardiograph, heart rate, blood alcohol content, respiratory rate, and glucose level.

14. The system of claim 8, wherein the received second authentication data is encrypted prior to transmission to the remote server.

15. A non-transitory computer-readable storage medium, having embodied thereon a program executable by a processor to perform the method of claim 1.
